(19) United States
(12) Patent Application Publication
(10) Pub. No.: US 2026/0175048 A1
(43) Pub. Date: Jun. 25, 2026

(54) SYSTEMS AND METHODS FOR DETERMINING A LOCATION OF A GROSS TARGET VOLUME OF A PATIENT

(71) Applicant: Siemens Healthineers International AG, Steinhausen (CH)

(72) Inventors: Yao-Jen Chang, Princeton, NJ (US); Ankur Kapoor, Plainsboro, NJ (US); Simon Arberet, Princeton, NJ (US); Florin-Cristian Ghesu, Baiersdorf (DE); Anand Santhanam, Monmouth Junction, NJ (US); Ali Kamen, Skillman, NJ (US)

(21) Appl. No.: 18/999,536

(22) Filed: Dec. 23, 2024

(51) Int. Cl. A61N 5/10; G06T 7/00

(52) U.S. Cl. CPC A61N 5/1036; A61N 5/1049; G06T 7/0012; G06T 2207/10; G06T 2207/20076; G06T 2207/30096

(57) ABSTRACT

Provided herein are systems for determining a location of a gross target volume of a patient. In some examples, systems can include one or more processors that are configured to obtain image data associated with a plurality of images of a lesion of a patient. For each image, the one or more processors can be configured to backproject points representing the lesion into the 3D space to determine a plurality of distribution confidence values for a subset of voxels within the three-dimensional space. The one or more processors can be configured to determine a three-dimensional confidence distribution based on confidence values from the plurality of distribution confidence values corresponding to each voxel of the 3D space and determine a position of the lesion within the 3D space based on the 3D confidence distribution.

### TECHNICAL FIELD

This application relates generally to systems and methods for determining a location of a gross target volume (GTV) of a patient and, in some embodiments, to systems and methods for determining a location of a gross target volume of a patient through three-dimensional (3D) lesion position reconstruction.

### BACKGROUND

Radiotherapy (also referred to as “radiotherapy” or “RT”) involves the delivery of radiation (energy) to targets within the body of a patient during treatment. For example, a linear accelerator (LINAC) can be configured to move relative to a patient in accordance with an RT treatment plan to multiple control points and deliver energy to target tissue (e.g., tumors, lesions, etc.) to treat cancer located within the body of the patient. This can allow for the targeted delivery of energy toward the target tissue, with the goal of reducing or eliminating the target tissue without affecting the surrounding tissue.

But conventional methods for implementing treatment plans to treat a patient can be difficult to execute in a controlled manner. For example, a patient positioned relative to a LINAC can move unintentionally when breathing or as a result of reflexive movement. This can cause a GTV to move relative to the LINAC, possibly resulting in the subsequent delivery of energy to healthy, non-target tissue. To reduce the chances of this occurring, patients being treated can be instructed to hold their breath as energy is delivered and coordinated operation of the LINAC by clinicians monitoring the patient to deliver energy while the patient is able to comply with the instruction. And techniques that involve monitoring the patient's movement based on external features of the patient can be subject to “drift” whereby the position of the tumor or lesion moves over time relative to the external features of the patient, resulting in misalignment between the LINAC and the patient.

### SUMMARY

For the aforementioned reasons, there is a need for systems and methods that improve on the process for determining the location of GTVs of patients when delivering energy to the patient as part of an RT treatment plan. Moreover, there is a need for a more innovative approach to determining the location of the GTV in real-time as energy is delivered to the patient in accordance with a predetermined treatment plan.

The methods and systems discussed herein address the challenge of reconstructing precise 3D lesion positions in radiotherapy, where x-ray imaging provides only 2D projections, leaving the depth along the sightline unobservable and complicating metric localization. This limitation is particularly critical for Beam-Eye View (BEV) positioning, as the radiation beam is typically placed at a 90-degree angle to the x-ray imager, rendering one major lesion coordinate unobservable. Existing and conventional solutions have been proven to be inadequate due to lesion drift or imperfect correlations between surrogate and lesion motion, often necessitating treatment pauses for re-imaging or expanded radiation areas to ensure coverage. To overcome these issues, the methods and systems discussed herein provide for probabilistic 3D lesion position reconstruction by acquiring x-ray images during treatment, deriving 2D lesion position distributions, fusing these distributions from selected views to generate 3D position distributions, and estimating BEV positions with uncertainty. This approach enhances localization accuracy and treatment adaptability by addressing the limitations of traditional 2D imaging and providing reliable, probabilistic modeling for radiotherapy.

The methods and systems discussed herein offer several technical benefits, including enhanced accuracy through the fusion of 2D detection distribution results for a more precise 3D lesion position reconstruction compared to traditional methods. These methods and systems improve treatment adjustments by allowing better targeting and potentially reducing treatment interruptions. Additionally, the methods and systems discussed herein account for uncertainties due to patient movement or breathing, providing a robust solution for real-time radiotherapy. These benefits collectively contribute to more effective and efficient cancer treatment through radiotherapy.

The methods and systems discussed herein can estimate the uncertainty of the position of the GTV relative to a radiotherapy system (e.g., a LINAC and/or the like) when delivering energy to the GTV. For example, as X-ray images are generated to monitor a GTV during treatment of a patient using a LINAC, the X-ray images (which are two-dimensional) can be projected (e.g., “backprojected”) into a 3D representation of the environment in which the LINAC and the patient are located. This can allow for reconstruction of the location of the GTV despite the distance along the sight of view of any given X-ray being not observable and subsequent determination of the GTV relative to the LINAC. Furthermore, the embodiments described herein can allow for the placement of the imaging device generating the X-ray images at a 90-degree angle of separation from the LINAC's beam path. This, in turn, can allow for the generation of increasingly precise projection of the GTV at the BEV. And by determining the 3D position of the GTV as described herein, the need for complex computing devices and/or systems to monitor external features of the patient (e.g., motion capture devices and/or systems) and register the position of the patient (and inferred position of the GTV) to the LINAC can be reduced or eliminated, saving computational resources (e.g., processing and memory) that would otherwise be involved in determining the position of the GTV during treatment of the patient.

In an embodiment, a system for determining a location of a gross target volume (GTV) of a patient is disclosed. The system can include one or more processors configured to obtain image data associated with a plurality of images of a lesion of a patient positioned in a three-dimensional (3D) space. The plurality of images can be captured from a plurality of imaging devices positioned about the patient. the one or more processors can be configured to, for each image of the plurality of images, backproject points representing the lesion into the 3D space to determine a plurality of distribution confidence values for a subset of voxels within the three-dimensional space. In some implementations, the one or more processors can be configured to determine a three-dimensional confidence distribution based on confidence values from the plurality of distribution confidence values corresponding to each voxel of the 3D space; and determine a position of the lesion within the 3D space based on the 3D confidence distribution.

In some aspects, the one or more processors are further configured to: determine a position of a radiotherapy system configured to deliver energy relative to at least a portion of the 3D space. The one or more processors can be configured to generate a beam-eye view of the patient relative to the radiotherapy system based on the position of the radiotherapy system and the position of the lesion within the 3D space. In some aspects, the one or more processors configured to obtain the image data can be configured to: obtain the image data from the plurality of imaging devices that are positioned in fixed relation to the 3D space, and in response to registration of a radiotherapy system with the 3D space or the patient, generate a projection of the estimated lesion position represented by distribution confidence values at a beam-eye view of the patient relative to the radiotherapy system. In aspects, the one or more processors configured to obtain the image data can be configured to: obtain the image data from the plurality of imaging devices that are positioned in fixed relation to a radiotherapy system. The one or more processors can be configured to, in response to registration of a radiotherapy system with the 3D space or the patient, generate a beam-eye view of the distribution of confidence values of the estimated lesion position of the patient relative to the radiotherapy system.

In some aspects, the one or more processors configured to determine the three-dimensional confidence distribution based on confidence values can be configured to: for each voxel of the subset of voxels, multiply the confidence values corresponding to each voxel established in response to backprojecting the points representing the lesion to establish a confidence value for each voxel, and normalize the confidence values for each voxel. The one or more processors configured to determine the three-dimensional confidence distribution based on confidence values can be configured to: determine a plurality of Gaussian distributions based on the confidence values corresponding to each voxel and determine the three-dimensional confidence distribution based on the Gaussian distribution of the confidence values. In aspects, the one or more processors can be further configured to: compare the position of the lesion within the 3D space to a treatment plan; and generate a set of one or more control signals to adjust a position, a power level, or a leaf sequence of a radiotherapy system with the 3D space, the one or more control signals configured to cause the radiotherapy system to deliver energy to the lesion in accordance with the treatment plan.

In embodiments, a method is described, including: obtaining, by one or more processors, image data associated with a plurality of images of a lesion of a patient positioned in a three-dimensional (3D) space, where the plurality of images are captured from a plurality of imaging devices positioned about the patient; and for each image of the plurality of images, backprojecting, by the one or more processors, points representing the lesion into the 3D space to determine a plurality of distribution confidence values for a subset of voxels within the three-dimensional space. The method can include determining, by the one or more processors, a three-dimensional confidence distribution based on confidence values from the plurality of distribution confidence values corresponding to each voxel of the 3D space. In some implementations, the method can include determining, by the one or more processors, a position of the lesion within the 3D space based on the 3D confidence distribution.

In some aspects, the method can include determining, by the one or more processors, a position of a radiotherapy system configured to deliver energy relative to at least a portion of the 3D space; and generating, by the one or more processors, a beam-eye view of the distribution of confidence values of the estimated lesion position of the patient relative to the radiotherapy system based on the position of the radiotherapy system and the position of the lesion within the 3D space. Obtaining the image data can include obtaining, by the one or more processors, the image data from the plurality of imaging devices that are positioned in fixed relation to the 3D space. In some implementations, in response to registration of a radiotherapy system with the 3D space or the patient, the method can include generating, by the one or more processors, a beam-eye view of the distribution of confidence values of the estimated lesion position of the patient relative to the radiotherapy system.

In some aspects, obtaining the image data can include obtaining, by the one or more processors, the image data from the plurality of imaging devices that are positioned in fixed relation to a radiotherapy system. In response to registration of a radiotherapy system with the 3D space or the patient, the method can include generating, by the one or more processors, a beam-eye view of the distribution of confidence values of the estimated lesion position of the patient relative to the radiotherapy system. In some implementations, determining the three-dimensional confidence distribution based on confidence values can include: for each voxel of the subset of voxels, multiplying, by the one or more processors, the confidence values corresponding to each voxel established in response to backprojecting the points representing the lesion to establish a confidence value for each voxel, and normalizing, by the one or more processors, the confidence values for each voxel. In aspects, determining the three-dimensional confidence distribution based on confidence values can include determining, by the one or more processors, a plurality of Gaussian distributions based on the confidence values corresponding to each voxel, and determining, by the one or more processors, the three-dimensional confidence distribution based on the Gaussian distribution of the confidence values.

In some aspects, the method can include comparing, by the one or more processors the position of the lesion within the 3D space to a treatment plan, and generating, by the one or more processors, a set of one or more control signals to adjust a position, a power level, or a leaf sequence of a radiotherapy system with the 3D space, the one or more control signals configured to cause the radiotherapy system to deliver energy to the lesion in accordance with the treatment plan.

In yet another embodiment, a non-transitory computer-readable medium storing instructions thereon is disclosed. The instructions, when executed by one or more processors, can cause the one or more processors to: obtain image data associated with a plurality of images of a lesion of a patient positioned in a three-dimensional (3D) space, where the plurality of images are captured from a plurality of imaging devices positioned about the patient. In some implementations, for each image of the plurality of images, the instructions can cause the one or more processors to backproject points representing the lesion into the 3D space to determine a plurality of distribution confidence values for a subset of voxels within the three-dimensional space. The instructions can then cause the one or more processors to determine a three-dimensional confidence distribution based on confidence values from the plurality of distribution confidence values corresponding to each voxel of the 3D space and determine a position of the lesion within the 3D space based on the 3D confidence distribution.

In some aspects, the instructions can further cause the one or more processors to: determine a position of a radiotherapy system configured to deliver energy relative to at least a portion of the 3D space, and generate a beam-eye view of the distribution of confidence values of the estimated lesion position of the patient relative to the radiotherapy system based on the position of the radiotherapy system and the position of the lesion within the 3D space. The instructions that cause the one or more processors to obtain the image data can cause the one or more processors to: obtain the image data from the plurality of imaging devices that are positioned in fixed relation to the 3D space, and in response to registration of a radiotherapy system with the 3D space or the patient, generate a beam-eye view of the distribution of confidence values of the estimated lesion position of the patient relative to the radiotherapy system. In some implementations, the instructions that cause the one or more processors to obtain the image data can cause the one or more processors to: obtain the image data from the plurality of imaging devices that are positioned in fixed relation to a radiotherapy system; and in response to registration of a radiotherapy system with the 3D space or the patient, generate a beam-eye view of the distribution of confidence values of the patient relative to the radiotherapy system.

In aspects, the instructions that cause the one or more processors to determine the three-dimensional confidence distribution can cause the one or more processors to: for each voxel of the subset of voxels, multiply the confidence values corresponding to each voxel established in response to backprojecting the points representing the lesion to establish a confidence value for each voxel, and normalize the confidence values for each voxel. The instructions that cause the one or more processors to determine the three-dimensional confidence distribution based on confidence values can cause the one or more processors to: determine a plurality of Gaussian distributions based on the confidence values corresponding to each voxel; and determine the three-dimensional confidence distribution based on the Gaussian distribution of the confidence values.

### BRIEF DESCRIPTION OF THE DRAWINGS

Non-limiting embodiments of the present disclosure are described by way of example with reference to the accompanying figures, which are schematic and are not intended to be drawn to scale. Unless indicated as representing the background art, the figures represent aspects of the disclosure.

FIG. 1 illustrates a diagram of a system for determining a location of a gross target volume of a patient, according to an embodiment.

FIG. 2 illustrates a flow diagram of a process for determining a location of a gross target volume of a patient, according to an embodiment.

FIG. 3 illustrates an example three-dimensional space in which a plurality of imaging devices is positioned to capture images, according to an embodiment.

FIGS. 4A and 4B, illustrate example views including isometric views and side views of a position and/or orientation of a GTV as determined in accordance with one or more of the techniques described herein, according to an embodiment.

### DETAILED DESCRIPTION

Reference will now be made to the illustrative embodiments depicted in the drawings, and specific language will be used here to describe the same. It will nevertheless be understood that no limitation of the scope of the claims or this disclosure is thereby intended. Alterations and further modifications of the inventive features illustrated herein, and additional applications of the principles of the subject matter illustrated herein, which would occur to one skilled in the relevant art and having possession of this disclosure, are configured to be considered within the scope of the subject matter disclosed herein. Other embodiments can be used, or other changes can be made without departing from the spirit or scope of the present disclosure. The illustrative embodiments described in the detailed description are not meant to be limiting of the subject matter presented.

The systems and methods described, as well as the techniques they implement, improve conventional RT treatment planning and implementation techniques. More specifically, in embodiments, systems described herein can be implemented to determine a location of a gross target volume (GTV) of a patient during treatment of the patient. Such systems can include one or more processors that are configured to obtain image data associated with a plurality of images of a lesion of a patient in a three-dimensional (3D) space, where the plurality of images is captured from a plurality of imaging devices positioned about the patient. The systems can, for each image of the plurality of images, backproject points representing the lesion into the 3D space to determine a plurality of distribution confidence values for a subset of voxels within the three-dimensional space. The system can then determine a three-dimensional confidence distribution based on confidence values from the plurality of distribution confidence values corresponding to each voxel of the 3D space and determine a position of the lesion within the 3D space based on the 3D confidence distribution.

By virtue of the implementation of the techniques described herein, the position of the GTV relative to a radiotherapy system (e.g., a LINAC and/or the like) when delivering energy to the GTV. For example, as X-ray images are generated to monitor a GTV during the delivery of RT treatment, a computing device (e.g., an analytics server as described herein) can be configured to obtain the X-ray images and backproject the visual representation of the GTV into a 3D representation of the environment in which the LINAC and the patient are located. By using multiple, two-dimensional images, different representations of the GTV from different points of view can be used to reconstruct the GTV with varying degrees of certainty per-voxel. In addition to the benefits stated earlier, these techniques can allow for real-time acquisition of images to determine a location (e.g., a pose) of the GTV relative to the patient as opposed to other techniques (e.g., the use of 3D scans) which can require lengthy scanning processes. Further, the use of two-dimensional images can allow for reduced exposure to radiation by the patient during treatment. And 2D imaging techniques, such as planar X-rays, typically require less time to acquire and process than three-dimensional methods, speeding up the scanning process and allowing for shorter treatment setup times, allowing for more efficient patient flow in clinical settings.

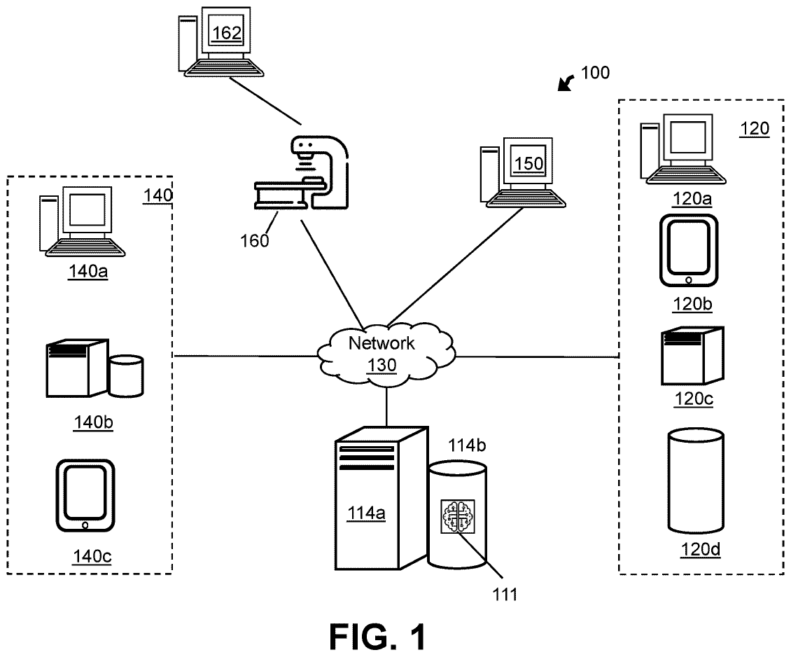

FIG. 1 illustrates components of a system 100 for determining a location of a gross target volume (GTV) of a patient, according to an embodiment. The system 100 can include an analytics server 114a, system database 114b, a treatment planning system 111, electronic data sources 120a-d (each referred to individually as an electronic data source 120 and collectively electronic data sources 120, unless stated otherwise), end-user devices 140a-c (each referred to individually as an end user device 140 and collectively as end-user devices 140, unless stated otherwise), an administrator computing device 150, a medical device 160, and medical device computer(s) 162. Various components depicted in FIG. 1 can belong to a radiotherapy clinic at which patients can receive radiotherapy treatment, in some cases via one or more radiotherapy machines (e.g., medical device 160) located within the clinic. The system 100 is not confined to the components described herein and can include additional or other components, not shown for brevity, which are configured to be considered within the scope of the embodiments described herein.

The above-mentioned components can be connected to each other through a network 130. Examples of the network 130 can include, but are not limited to, private or public local-area-networks (LAN), wireless LAN (WLAN) networks, metropolitan area networks (MAN), wide-area networks (WAN), and the Internet. The network 130 can include wired or wireless communications according to one or more standards or via one or more transport mediums. The communication over the network 130 can be performed in accordance with various communication protocols such as Transmission Control Protocol and Internet Protocol (TCP/IP), User Datagram Protocol (UDP), and IEEE communication protocols. In one example, the network 130 can include wireless communications according to Bluetooth specification sets or another standard or proprietary wireless communication protocol. In another example, the network 130 can also include communications over a cellular network, including, e.g., a GSM (Global System for Mobile Communications), CDMA (Code Division Multiple Access), and EDGE (Enhanced Data for Global Evolution) network.

The analytics server 114a can be any computing device comprising a processor and non-transitory machine-readable storage capable of executing the various tasks and processes described herein. The analytics server 114a can employ various processors such as central processing units (CPU) and graphics processing unit (GPU), among others. Non-limiting examples of such computing devices can include workstation computers, laptop computers, server computers, and the like. While the system 100 includes a single analytics server 114a, the analytics server 114a can include any number of computing devices operating in a distributed computing environment, such as a cloud environment.

The analytics server 114a can generate and display an electronic platform configured to use a treatment planning system 111 for receiving patient information and inputs from users (e.g., clinicians) such as utility functions and updated utility functions described herein and outputting the results of execution of the treatment planning system 111. The electronic platform can include graphical user interfaces (GUI) displayed by display devices of one or more electronic data sources 120, the end-user devices 140, the medical device 160, or the administrator computing device 150. An example of the electronic platform generated and hosted by the analytics server 114a can be a web-based application or a website configured to be displayed on different electronic devices, such as mobile devices, tablets, personal computers, and the like.

The information displayed by the electronic platform can include, for example, input elements to receive data associated with a patient being treated, synchronize one or more sensors, and display results of predictions produced by the treatment planning system 111. For instance, the analytics server 114a can execute the treatment planning system 111 (e.g., a system such as a treatment planner that is configured or trained to generate beam configurations that are usable to configure the medical device 160 when treating a patient, as described herein). The analytics server 114a can then display the results for a clinician or directly revise one or more operational attributes of the medical device 160.

The electronic data sources 120 can be any computing device comprising a processor and non-transitory machine-readable storage capable of executing the various tasks and processes described herein. For example, the electronic data sources 120 can represent various computing devices that contain, retrieve, or access data associated with a medical device 160, such as data associated with operational information of currently or previously performed radiotherapy treatments (e.g., electronic log files or electronic configuration files), data associated with current or previously monitored patients (e.g., computed tomography (CT) scans, magnetic resonance imaging (MRI) scans, tumor locations, deformation information, or the like) or participants in a study, or the like. For instance, the analytics server 114a can use the clinic computer 120a, medical professional device 120b, server 120c (associated with a clinician or a clinic), and database 120d (associated with the clinician or the clinic) to retrieve/receive data associated with the medical device 160. The analytics server 114a can retrieve the data from the end-user devices 140, generate a dataset, and use the dataset to configure the treatment planning system 111 (e.g., models implemented by the treatment planning system 111 or the like). The analytics server 114a can execute various algorithms to translate raw data received/retrieved from the electronic data sources 120 into machine-readable objects that can be stored and processed by other analytical processes as described herein.

End-user devices 140 can be any computing device comprising a processor and a non-transitory machine-readable storage medium capable of performing the various tasks and processes described herein. Non-limiting examples of an end-user device 140 can be a workstation computer, laptop computer, tablet computer, or server computer. In operation, various users such as clinicians as described herein can use end-user devices 140 to access the GUI operationally managed by the analytics server 114a or otherwise the results of the execution of the treatment planning system 111. Specifically, the end-user devices 140 can include clinic computer 140a, clinic server 140b, and a medical professional device 140c. Even though referred to herein as “end-user” devices, these devices cannot always be operated by end-users. For instance, the clinic server 140b cannot be directly used by an end user. However, the results stored on the clinic server 140b can be used to populate various GUIs accessed by an end user via the medical professional device 140c. In some embodiments, the end-user device 140 can be associated with one or more clinicians that are associated with the generation of one or more treatment plans (e.g., involved in preparing the one or more treatment plans) for patients.

The administrator computing device 150 can represent a computing device operated by a system administrator. The administrator computing device 150 can be configured to display radiotherapy treatment attributes generated by the analytics server 114a (e.g., various analytic metrics determined during training of one or more machine learning models or systems); monitor various treatment planning systems 111 utilized by the analytics server 114a, electronic data sources 120, or end-user devices 140; review feedback; or facilitate training or retraining (calibration) of the treatment planning system 111 that are maintained by the analytics server 114a.

In some embodiments, the medical device 160 can be a diagnostic imaging device or a treatment delivery device (also referred to as a radiotherapy system). For example, the medical device 160 can include one or more computed tomography (CT) scanners such as a cone-beam CT (CBCT) scanner, a linear accelerator (LINAC) such as the Varian® TrueBeam® LINAC, proton beam therapy systems (referred to as proton beam systems) that uses accelerated protons to deliver precise radiotherapy to tumors, or other similar devices configured to transmit energy toward targeted tissue (referred to as gross target volumes) associated with a patient and, in some cases, measure the energy transferred to ward the targeted tissue. The medical device 160 can also include one or more sensors configured to monitor the patient being treated. That is, the medical device 160 or the analytics server 114a can be communicating with various sensors that can monitor a patient's external biological signals. Non-limiting examples of the sensors can include 3D surfacing mechanisms, and optical (or other) sensors configured to monitor the patient's movements (e.g., how the patient is moving or breathing. In some embodiments, the medical device 160 can receive data associated with a treatment plan from the medical device computer(s) 162 that cause the medical device 160 to operate in accordance with the treatment plan.

The treatment planning system 111 can be stored in the system database 114b. The treatment planning system 111 can be trained using data received/retrieved from the electronic data sources 120 and can be executed using data received from the end-user devices, the medical device 160, or the sensor 163. In some embodiments, the treatment planning system 111 can reside within a data repository local or specific to a clinic. In various embodiments, the treatment planning system 111 can use one or more deep learning engines to develop a treatment plan for a patient using radiotherapy. For instance, the analytics server 114a can transmit patient attributes from the sensor 163 and execute the treatment planning system 111 accordingly. The analytics server 114a can then display the results on one or more end-user devices 140. In some embodiments, the analytics server 114a can change one or more configurations of the medical device 160 based on the results predicted by the treatment planning system 111.

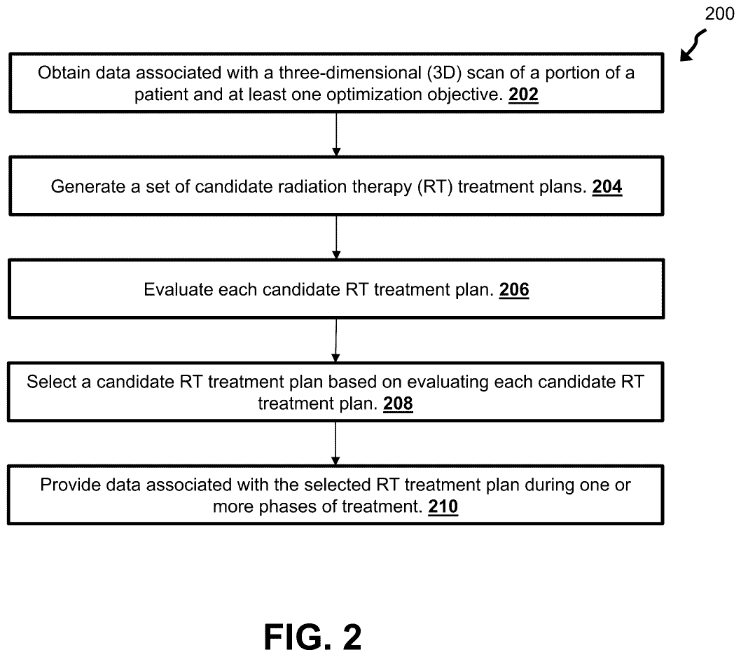

Referring to FIG. 2, illustrated is a flow diagram of a process 200 for determining a location of a lesion (and/or a gross target volume (GTV) including one or more lesions) of a patient. The process 200 includes operations 202-208. However, other embodiments can include additional or alternative operations or can omit one or more operations altogether. The process 200 is described as being executed by an analytics server, which can be the same as, or similar to, the analytics server 114a described in FIG. 1. However, one or more steps of the process 200 can be executed by any number of computing devices operating in the distributed computing system described in FIG. 1. For instance, one or more computing devices can locally perform part, or all of the operations described in FIG. 2.

At operation 202, the analytics server can obtain image data associated with a plurality of images of a lesion of a patient. For example, the analytics server can obtain the image data from a plurality of imaging devices such as X-ray machine and/or the like. In some embodiments, the analytics server can obtain the image data from the plurality of imaging devices, where the plurality of imaging devices is positioned relative to a patient. In this example, the plurality of imaging devices can be positioned relative to the patient along with a medical device (e.g., that is the same as, or similar to, the medical device 160 of FIG. 1) such as a LINAC that is being moved to multiple control points to deliver energy to the patient. While the concepts of the present disclosure are discussed with respect to a LINAC, it will be understood that different types of medical devices (e.g., proton beam systems, etc.) are contemplated as being used instead of, or in addition to, the LINAC.

In one example, during RT treatment, a patient can be positioned in relation to a gantry of a LINAC that supports a magnetron or a klystron generating high-energy X-rays for delivery to the lesion of the patient. The LINAC can include a multi-leaf collimator (MLC) that is positioned along a treatment beam path (beam path) that is configured to shape the high energy X-rays as they are directed toward the lesion of the patient. In some embodiments, the plurality of imaging devices can be positioned such that they target at least a portion of the patient including a lesion targeted as part of a treatment plan for imaging to allow for reconstruction of the environment in which the patient is located. For example, during treatment of the patient, the LINAC can be moved to a plurality of control points (e.g., 3D poses within the 3D space that the patient, the LINAC, and the imaging devices are located in) and configured to generate and transmit energy toward the patient at each control point. At each control point, the leaves of the LINAC's MLC can shape the X-rays so as to optimize energy delivery and conformance with the lesion of the patient, while minimizing the delivery of energy outside of the lesion (e.g., to one or more OARs). In some embodiments, a set of control points, MLC leaf configurations, and power levels at which energy is to be delivered can be established by a treatment plan generated prior to the procedure. As will be understood, the analytics server can be configured to perform operations to control the operation of the LINAC as described herein.

In some embodiments, the analytics server can obtain the image data based on one or more view selections. For example, the analytics server can obtain the image data based on execution of a nearest neighbor algorithm. In this example, the analytics server can select views from the last k frames in time in addition to a current view. This can be appropriate when implemented in coordination with efforts of the patient to hold their breath and not move, where lesion motion is assumed to be static or very small even though its precise location is unpredictable. In another example, the analytics server can obtain the image data based on execution of a sequential stereo algorithm. In this example, the analytics server can select images based on factors such as stereo separation, 3D distance from the current ray, and sufficient clustering at the approximate crossing point with other rays. In yet another example, the analytics server can obtain the image data based on execution of a surrogate-assisted view selection algorithm. For example, for treatments conducted with free breathing, external surrogates can be used to infer the target motion based on the observed surrogate motion. In this example, the analytics server can select frames with similar surrogate readings as the one from the current frame.

In some embodiments, the analytics server can obtain image data at one or more points in time. For example, the imaging devices can be configured to capture images of the patient before, during, or after the delivery of energy to the lesions in the lesion of the patient to target the lesion. This image data can be generated and obtained by the analytics server at one or more discrete points in time, continuously, etc. In some embodiments, the plurality of images can be captured simultaneously such that each image of the plurality of images represent a state of the patient (e.g., a portion of the patient including the lesion at a given point in time). The imaging devices can then transmit the image data to the analytics server to allow the analytics server to determine a for the lesion at the point in time as described herein.

In some embodiments, the imaging data can be generated by a plurality of imaging devices that are positioned in fixed relation to the 3D space in which the RT treatment is being performed. For example, each imaging device of the plurality of imaging devices can be located in fixed relation to the 3D space and configured to generate image data including a plurality of 2D images of the patient who may be positioned on a treatment couch to optimize energy delivery to the lesion of the patient. Prior to treatment, once the patient is positioned on the treatment couch, the plurality of imaging devices can be positioned (e.g., focused) such that they are directed toward a portion of the patient that includes the lesion being treated. The analytics server can then cause the plurality of imaging devices to capture images of the patient to allow for the determination of the location of the lesion relative to the 3D space and/or the LINAC. For example, the analytics server can initially register the pose of the LINAC with the 3D space along with the imaging devices. In response to registration of the LINAC and the imaging devices within the 3D space, the analytics server can obtain imaging data and generate (e.g., reconstruct) a beam-eye view of the lesion. Additionally, or alternatively, in response to registration of the LINAC and/or the imaging devices with the patient (e.g., with one or more points along the patient), the analytics server can obtain imaging data and generate a beam-eye view of the lesion.

At operation 204, for each image of the plurality of images, the analytics server can backproject points representing the lesion (e.g., the lesion) into the 3D space. In some embodiments, to generate the beam-eye view, the analytics server can backproject points representing the lesion into the 3D space. For example, the analytics server can process each image of the plurality of images represented by the imaging data. The analytics server can then determine (e.g., identify) points within the plurality of images as corresponding to the lesion and not corresponding to the lesion (e.g., representing other tissue, objects in proximity to the patient, etc.). As a result, the analytics server can determine (e.g., derive) a set of positions of the lesion for each image (e.g., X-ray image) represented by the imaging data.

Using these sets of 2D positions, the analytics server can backproject the points representing the lesion into a virtual representation of the 3D space in which the patient is located. For example, the analytics server can determine corresponding 3D coordinates within the 3D space of points represented in in each image (e.g., each 2D image) of the plurality of images. In one example, the analytics server can execute a camera model which is configured to determine a correspondence between the 2D points from the plurality of images within the 3D space. This correspondence can be determined based on intrinsic parameters (e.g., focal length and/or the like) and extrinsic parameters (e.g., the position, orientation, and/or pose of the imaging devices in the 3D space). The projection from the 2D images to the 3D space can also involve normalizing the 2D point, constructing a ray from the camera through the point, and finding the intersection of this ray with a known plane in 3D space.

In some embodiments, in response to backprojecting the 2D points into the virtual representation of the 3D space, the analytics server can determine a plurality of distribution confidence values. For example, the analytics server can first backproject the 2D points into the virtual representation of the 3D space, assigning confidence values to each voxel associated with one or more points in one or more of the images represented by the imaging data. These confidence values can represent a likelihood that a given voxel corresponding to a point in at least one of the images further corresponds to an actual position of the lesion within the 3D space. As a result, the analytics server can associate one or more confidence values with one or more voxels that can allow the analytics server to determine a distribution of confidence values. In examples, the analytics server can also normalize the confidence values assigned to each voxel.

At operation 206, the analytics server can determine a 3D confidence distribution. In some embodiments, the analytics server can determine a 3D confidence distribution based on the confidence values from the plurality of distribution confidence values. For example, the analytics server can determine the 3D confidence distribution based on the confidence values assigned to each voxel in the 3D space. In one example, the analytics server can multiply the confidence values corresponding to each voxel to establish a confidence value (e.g., a combined confidence value) for each voxel. This process can be iteratively repeated until all of the voxels associated with at least one voxel are established.

The analytics server can then normalize the confidence value for each value. For example, the analytics server can update the confidence values in the 3D confidence distribution in accordance with a Gaussian distribution. In this example, the confidence values in the 3D distribution can be normalized based on a Gaussian distribution by mean centering and scaling. This process can involve subtracting the mean from each value to center the distribution around zero. Each centered confidence value can then be divided by its standard deviation to scale the confidence values. The result can include a set of confidence values for each voxel within the virtual representation of the 3D space that follows a standard normal distribution with a mean of 0 and a standard deviation of 1 in each dimension.

At operation 208 the analytics server can determine a position of the lesion within the 3D space based on the 3D confidence distribution. For example, the analytics server can determine a position of the lesion within the 3D space based on the 3D confidence distribution. In examples, the analytics server can determine a position and/or an orientation (e.g., a pose) of the lesion within the 3D space based on the confidence values from the 3D confidence distribution. In an example, the analytics server can determine that the lesion occupies one or more voxels based on the confidence value corresponding to that voxel satisfying a threshold value. The analytics server can then determine for each confidence value whether the lesion is located or not located at the corresponding voxel within the 3D space. In some embodiments, the analytics server can then determine a position of a LINAC relative to the lesion. For example, the analytics server can determine a position and/or orientation of the LINAC based on registration of the LINAC with the 3D space. Additionally, or alternatively, the analytics server can determine the position and/or orientation of the LINAC based on registration of the LINAC with the patient.

In some embodiments, the analytics server can generate a beam-eye view of the lesion of the patient (e.g., of the lesion located within the patient that is represented by the images as if visualized from the beam source). For example, the analytics server can generate a beam-eye view of the patient relative to a portion of the LINAC. In one example, the analytics server can generate the beam-eye view based on (e.g., in response to the analytics server determining) the position and/or orientation of the lesion and the LINAC within the 3D space. The analytics server can then project the position and/or orientation of the lesion and the LINAC within the 3D space to generate the beam-eye view. The beam-eye view can represent the relative position and/or orientation of the lesion relative to a component (e.g., the MLC) of the LINAC by providing a visual representation of the treatment beam's path through the patient's anatomy when targeting the lesion. In some embodiments, the beam-eye view can additionally be generated by the analytics server based on reconstructions created from computed tomography (CT) scans of the patient (e.g., generated prior to the initiation of the RT treatment).

In some embodiments, the analytics server can compare the position of the lesion within the 3D space with a treatment plan. For example, the analytics server can compare the position of the lesion relative to the beam-eye view and compare the intersection of the beam from the beam-eye view to the treatment plan. In this example, the analytics server can then generate a set of one or more control signals to adjust a position of the LINAC (e.g., of the MLC) in response to determining a difference between the intersection of the beam with the lesion relative to a planned intersection of the beam with the lesion as represented in the treatment plan. These control signals can be adjusted relative to the control signals defined by the treatment plan to correct for the deviation so that the deviation is below a threshold amount (e.g., within a predetermined tolerance). The analytics server can then configure the control signal to adjust the position, a power level, a leaf sequence, etc., to control operation of the LINAC in the 3D space when delivering energy to the lesion in accordance with the treatment plan and transmit the control signals to the LINAC in accordance with the RT treatment plan. It will be understood that, at each point in time during the RT treatment plan, the operations described herein can be iteratively repeated until the treatment plan is complete.

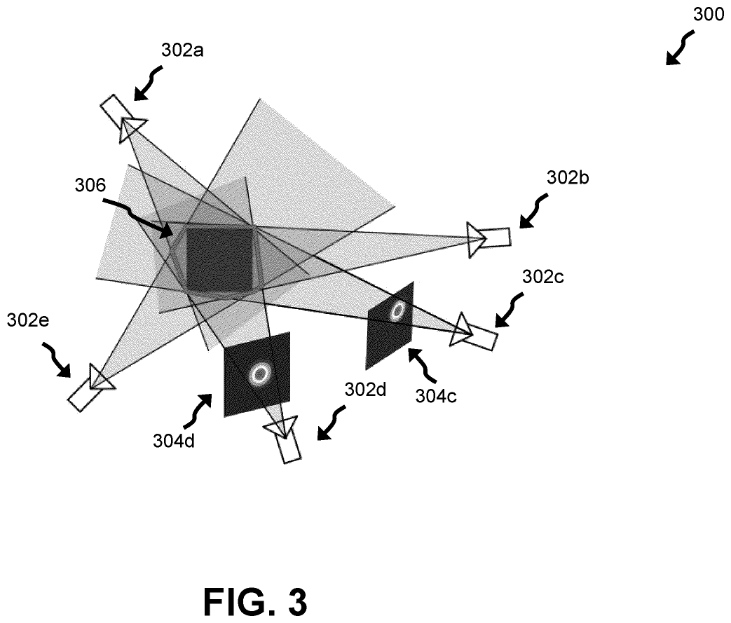

Referring to FIG. 3, illustrated is an example 3D space 300 in which a plurality of imaging devices is positioned to capture images, according to an embodiment. The 3D space 300 includes a plurality of imaging devices 302a-302e (referred to collectively as imaging devices 302 and individually as imaging device 302). The imaging devices 302 can include one or more X-ray machines and/or other similar devices capable of generating images of portion of tissue included in the patient.

The imaging devices 302 can be configured to generate one or more images such as image 304c and image 304d. While only two images 304c, 304d are illustrated, it will be understood that each imaging device 302 can be configured to generate one or more images as described herein (referred to collectively as images 304). In use, the imaging devices 302 can transmit energy along corresponding fields-of-view 306a-306c toward the lesion of the patient, and that energy can be captured by corresponding image receptors (not explicitly illustrated for purposes of clarity). As a result, some of the energy from the X-rays can be absorbed by the tissue of the patient in a region 308 and the remaining energy can be absorbed by the corresponding image receptors. The images (including images 304c, 304d) can then be used as described herein to backproject the points within the images associated with the lesion and determine a position and/or orientation of the lesion within the 3D space 300.

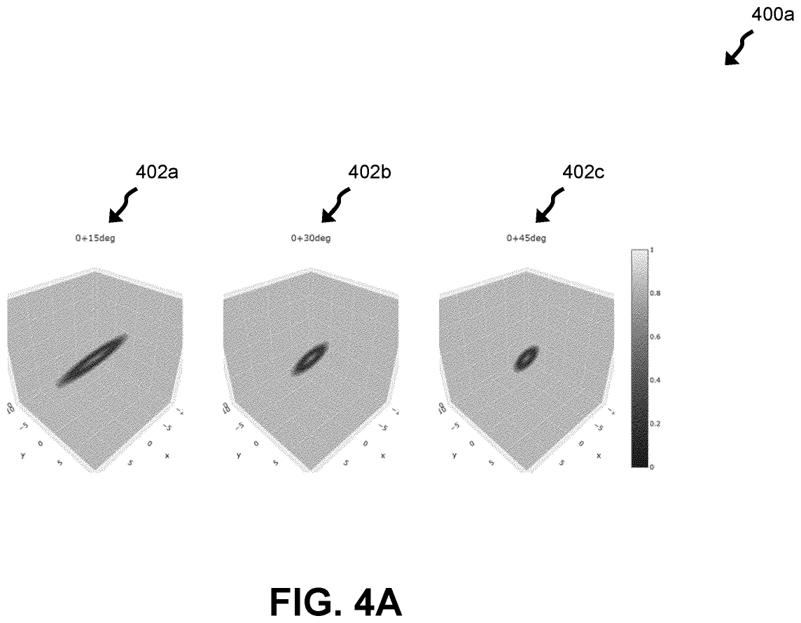

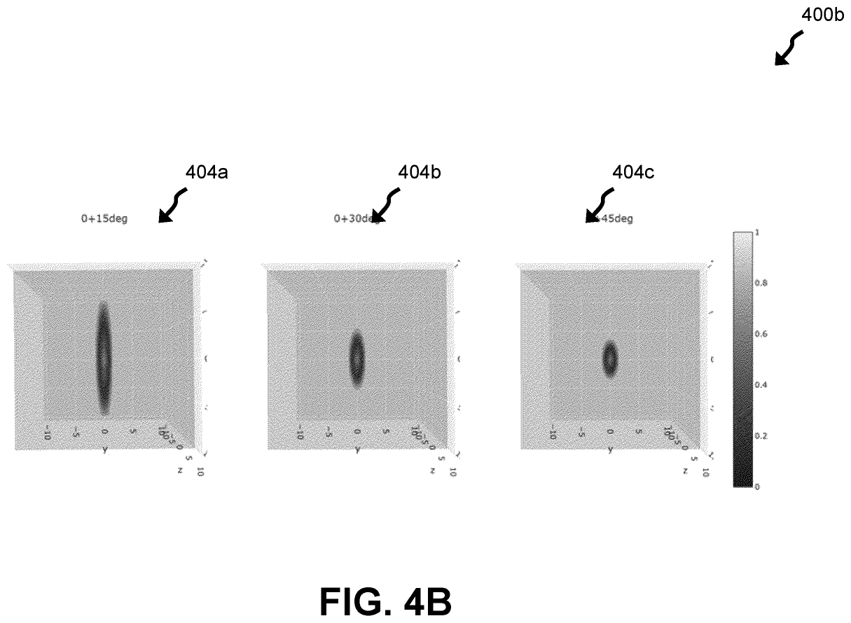

Referring now to FIGS. 4A and 4B, illustrated are example views 400 including isometric views 402a-402c and side views 404a-404c of a position and/or orientation of a lesion as determined in accordance with one or more of the techniques described herein. As shown, a lesion 406 can be imaged by one or more imaging devices as described in FIG. 2. The points representing the lesion 406 included in each image can then be backprojected to determine a plurality of confidence values. As show, the confidence values can be modeled as a gradient where darker regions of the lesion 406 represent areas where an analytics server (e.g., that is the same as, or similar to, the analytics server 114a of FIG. 1) estimates the lesion 406 to be located, each confidence value representing a likelihood that the lesion 406 is actually located as depicted. To model the lesion 406, the analytics server can determine a dense 3D grid surrounding the isocenter of the lesion 406, and then project each point from the images (e.g., that are the same as, or similar to, images 304c, 340d illustrated by FIG. 3) on the grid to the selected X-ray view to obtain a corresponding confidence value. The confidence values coming from multiple views can be fused by direct multiplication followed by global normalization. In FIG. 4A, three views 402a-402c are shown that include views from different angles of separation (e.g., views 402b and 402c) from a current reference view (e.g., view 402a) to form the 3D confidence distribution (also referred to as a 3D position distribution). As will be understood, smaller angle separations between views can cause larger uncertainty in the resulting 3D confidence distribution.

Additionally, or alternatively, the analytics server described herein can model the 3D confidence distribution using one or more Gaussian distributions. For example, Gaussian splatting (e.g., starting from sparse points generated during camera calibration and representing the scene with 3D Gaussians that preserve the properties of continuous volumetric radiance fields) can be implemented to adapt the parameters of Gaussians and dynamically determine the number of Gaussians to approximate the underlying 3D confidence distribution. In other examples, the analytics server can model the 3D confidence distribution as an implicit function. This function can be modeled using a deep neural network such as neural radiance field (NeRF) where each view ray is parameterized as a 5-D vector (x, y and z for observation position and q and f for 2D viewing direction) and the NeRF can outputs the color and density for each voxel along the ray. In this approach, the NeRF can generate volumetric representations of a 3D scene based on input images, while depth priors from monocular depth estimation networks are employed to guide the sampling process during optimization. By computing errors between the rendered images and the original input, confidence scores can be derived, which form a probability distribution reflecting the reliability of the reconstructed depths. This confidence distribution allows for adaptive sampling, prioritizing regions with higher certainty and effectively filtering out unreliable data, thereby enhancing the overall accuracy and robustness of the 3D reconstruction process.

In the examples described herein, once a 3D confidence distribution is obtained, an analytics server can generate views 404a-404c (also referred to as beam-eye views). These beam-eye views can represent a 2D projection of the 3D confidence distribution to a plane associated with a medical device (e.g., an MLC of a LINAC).

The presently-disclosed techniques will be better understood with reference to the following enumerated examples:
1. A system, comprising: one or more processors configured to: obtain image data associated with a plurality of images of a lesion of a patient positioned in a three-dimensional (3D) space, where the plurality of images are captured from a plurality of imaging devices positioned about the patient; for each image of the plurality of images, backproject points representing the lesion into the 3D space to determine a plurality of distribution confidence values for a subset of voxels within the three-dimensional space; determine a three-dimensional confidence distribution based on confidence values from the plurality of distribution confidence values corresponding to each voxel of the 3D space; and determine a position of the lesion within the 3D space based on the 3D confidence distribution.
2. The system of any of the previous examples, wherein the one or more processors are further configured to: determine a position of a radiotherapy system configured to deliver energy relative to at least a portion of the 3D space; and generate a beam-eye view of the lesion of the patient relative to the radiotherapy system based on the position of the radiotherapy system and the position of the lesion within the 3D space.
3. The system of any of the previous examples, wherein the one or more processors configured to obtain the image data are configured to: obtain the image data from the plurality of imaging devices that are positioned in fixed relation to the 3D space, and in response to registration of a radiotherapy system with the 3D space or the patient, generate a beam-eye view of the lesion of the patient relative to the radiotherapy system.
4. The system of any of the previous examples, wherein the one or more processors configured to obtain the image data are configured to: obtain the image data from the plurality of imaging devices that are positioned in fixed relation to a radiotherapy system; and in response to registration of a radiotherapy system with the 3D space or the patient, generate a beam-eye view of the lesion of the patient relative to the radiotherapy system.
5. The system of any of the previous examples, wherein the one or more processors configured to determine the three-dimensional confidence distribution based on confidence values are configured to: for each voxel of the subset of voxels, multiply the confidence values corresponding to each voxel established in response to backprojecting the points representing the lesion to establish a confidence value for each voxel, and normalize the confidence values for each voxel.
6. The system of any of the previous examples, wherein the one or more processors configured to determine the three-dimensional confidence distribution based on confidence values are configured to: determine a plurality of Gaussian distributions based on the confidence values corresponding to each voxel; and determine the three-dimensional confidence distribution based on the Gaussian distribution of the confidence values.
7. The system of any of the previous examples, wherein the one or more processors are further configured to: compare the position of the lesion within the 3D space to a treatment plan; and generate a set of one or more control signals to adjust a position, a power level, or a leaf sequence of a radiotherapy system with the 3D space, the one or more control signals configured to cause the radiotherapy system to deliver energy to the lesion in accordance with the treatment plan.
8. A method, comprising: obtaining, by one or more processors, image data associated with a plurality of images of a lesion of a patient positioned in a three-dimensional (3D) space, where the plurality of images are captured from a plurality of imaging devices positioned about the patient; for each image of the plurality of images, backprojecting, by the one or more processors, points representing the lesion into the 3D space to determine a plurality of distribution confidence values for a subset of voxels within the three-dimensional space; determining, by the one or more processors, a three-dimensional confidence distribution based on confidence values from the plurality of distribution confidence values corresponding to each voxel of the 3D space; and determining, by the one or more processors, a position of the lesion within the 3D space based on the 3D confidence distribution.
9. The method of any of the previous examples, further comprising: determining, by the one or more processors, a position of a radiotherapy system configured to deliver energy relative to at least a portion of the 3D space; and generating, by the one or more processors, a beam-eye view of the lesion of the patient relative to the radiotherapy system based on the position of the radiotherapy system and the position of the lesion within the 3D space.
10. The method of any of the previous examples, wherein obtaining the image data comprises: obtaining, by the one or more processors, the image data from the plurality of imaging devices that are positioned in fixed relation to the 3D space, and in response to registration of a radiotherapy system with the 3D space or the patient, generating, by the one or more processors, a beam-eye view of the lesion of the patient relative to the radiotherapy system.
11. The method of any of the previous examples, wherein obtaining the image data comprises: obtaining, by the one or more processors, the image data from the plurality of imaging devices that are positioned in fixed relation to a radiotherapy system; and in response to registration of a radiotherapy system with the 3D space or the patient, generating, by the one or more processors, a beam-eye view of the lesion of the patient relative to the radiotherapy system.
12. The method of any of the previous examples, wherein determining the three-dimensional confidence distribution based on confidence values comprises: for each voxel of the subset of voxels, multiplying, by the one or more processors, the confidence values corresponding to each voxel established in response to backprojecting the points representing the lesion to establish a confidence value for each voxel, and normalizing, by the one or more processors, the confidence values for each voxel.
13. The method of any of the previous examples, wherein determining the three-dimensional confidence distribution based on confidence values comprises: determining, by the one or more processors, a plurality of Gaussian distributions based on the confidence values corresponding to each voxel; and determining, by the one or more processors, the three-dimensional confidence distribution based on the Gaussian distribution of the confidence values.
14. The method of any of the previous examples, further comprising: comparing, by the one or more processors the position of the lesion within the 3D space to a treatment plan, the treatment plan; and generating, by the one or more processors, a set of one or more control signals to adjust a position, a power level, or a leaf sequence of a radiotherapy system with the 3D space, the one or more control signals configured to cause the radiotherapy system to deliver energy to the lesion in accordance with the treatment plan.
15. A computer program comprising instructions that when executed by a processor cause the processor to perform the steps of: obtaining image data associated with a plurality of images of a lesion of a patient positioned in a three-dimensional (3D) space, where the plurality of images are captured from a plurality of imaging devices positioned about the patient; for each image of the plurality of images, backprojecting points representing the lesion into the 3D space to determine a plurality of distribution confidence values for a subset of voxels within the three-dimensional space; determining a three-dimensional confidence distribution based on confidence values from the plurality of distribution confidence values corresponding to each voxel of the 3D space; and determining a position of the lesion within the 3D space based on the 3D confidence distribution.
16. The computer program of any of the previous examples, wherein the instructions further cause the processor to perform the steps of: determining a position of a radiotherapy system configured to deliver energy relative to at least a portion of the 3D space; and generating a beam-eye view of the lesion of the patient relative to the radiotherapy system based on the position of the radiotherapy system and the position of the lesion within the 3D space.
17. The computer program of any of the previous examples, wherein the instructions that cause the processor to obtain the image data cause the processor to perform the steps of: obtaining the image data from the plurality of imaging devices that are positioned in fixed relation to the 3D space, and in response to registration of a radiotherapy system with the 3D space or the patient, generating a beam-eye view of the lesion of the patient relative to the radiotherapy system.
18. The computer program of any of the previous examples, wherein the instructions that cause the processor to obtain the image data cause the processor to perform the steps of: obtaining the image data from the plurality of imaging devices that are positioned in fixed relation to a radiotherapy system; and in response to registration of a radiotherapy system with the 3D space or the patient, generating a beam-eye view of the patient relative to the radiotherapy system.
19. The computer program of any of the previous examples, wherein the instructions that cause the processor to determine the three-dimensional confidence distribution based on confidence values cause the processor to perform the steps of: for each voxel of the subset of voxels, multiplying the confidence values corresponding to each voxel established in response to backprojecting the points representing the lesion to establish a confidence value for each voxel, and normalize the confidence values for each voxel.
20. The computer program of any of the previous examples, wherein the instructions that cause the processor configured to determine the three-dimensional confidence distribution based on confidence values cause the processor to perform the steps of: determining a plurality of Gaussian distributions based on the confidence values corresponding to each voxel; and determining the three-dimensional confidence distribution based on the Gaussian distribution of the confidence values.

The various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans can implement the described functionality in varying ways for each particular application, but such implementation decisions should not be interpreted as causing a departure from the scope of this disclosure or the claims.

Embodiments implemented in computer software (e.g., computer programs, computer program products, etc.) can be implemented in software, firmware, middleware, microcode, hardware description languages, or any combination thereof. A code segment or machine-executable instructions can represent a procedure, a function, a subprogram, a program, a routine, a subroutine, a module, a software package, a class, or any combination of instructions, data structures, or program statements. A code segment can be coupled to another code segment or a hardware circuit by passing or receiving information, data, arguments, parameters, or memory contents. Information, arguments, parameters, data, etc., can be passed, forwarded, or transmitted via any suitable means, including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions can be stored as one or more instructions or code on a non-transitory computer-readable or processor-readable storage medium. The steps of a method or algorithm disclosed herein can be embodied in a processor-executable software module, which can reside on a computer-readable or processor-readable storage medium. A non-transitory computer-readable or processor-readable media includes both computer storage media and tangible storage media that facilitate the transfer of a computer program from one place to another. A non-transitory processor-readable storage media can be any available media that can be accessed by a computer. By way of example, and not limitation, such non-transitory processor-readable media can comprise RAM, ROM, EEPROM, CD-ROM or other optical disk storage, magnetic disk storage or other magnetic storage devices, or any other tangible storage medium that can be used to store desired program code in the form of instructions or data structures and that can be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm can reside as one or any combination or set of codes or instructions on a non-transitory processor-readable medium or computer-readable medium, which can be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein can be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. A system, comprising:
one or more processors configured to:
obtain image data associated with a plurality of images of a lesion of a patient positioned in a three-dimensional (3D) space, where the plurality of images are captured from a plurality of imaging devices positioned about the patient;
for each image of the plurality of images, backproject points representing the lesion into the 3D space to determine a plurality of distribution confidence values for a subset of voxels within the three-dimensional space;
determine a three-dimensional confidence distribution based on confidence values from the plurality of distribution confidence values corresponding to each voxel of the 3D space; and
determine a position of the lesion within the 3D space based on the 3D confidence distribution.

2. The system of claim 1, wherein the one or more processors are further configured to:
determine a position of a radiotherapy system configured to deliver energy relative to at least a portion of the 3D space; and
generate a beam-eye view of the lesion of the patient relative to the radiotherapy system based on the position of the radiotherapy system and the position of the lesion within the 3D space.

3. The system of claim 1, wherein the one or more processors configured to obtain the image data are configured to:
obtain the image data from the plurality of imaging devices that are positioned in fixed relation to the 3D space, and
in response to registration of a radiotherapy system with the 3D space or the patient, generate a beam-eye view of the lesion of the patient relative to the radiotherapy system.

4. The system of claim 1, wherein the one or more processors configured to obtain the image data are configured to:
obtain the image data from the plurality of imaging devices that are positioned in fixed relation to a radiotherapy system; and
in response to registration of a radiotherapy system with the 3D space or the patient, generate a beam-eye view of the lesion of the patient relative to the radiotherapy system.

5. The system of claim 1, wherein the one or more processors configured to determine the three-dimensional confidence distribution based on confidence values are configured to:
for each voxel of the subset of voxels, multiply the confidence values corresponding to each voxel established in response to backprojecting the points representing the lesion to establish a confidence value for each voxel, and
normalize the confidence values for each voxel.

6. The system of claim 1, wherein the one or more processors configured to determine the three-dimensional confidence distribution based on confidence values are configured to:
determine a plurality of Gaussian distributions based on the confidence values corresponding to each voxel; and
determine the three-dimensional confidence distribution based on the Gaussian distribution of the confidence values.

7. The system of claim 1, wherein the one or more processors are further configured to:
compare the position of the lesion within the 3D space to a treatment plan; and
generate a set of one or more control signals to adjust a position, a power level, or a leaf sequence of a radiotherapy system with the 3D space, the one or more control signals configured to cause the radiotherapy system to deliver energy to the lesion in accordance with the treatment plan.

8. A method, comprising:
obtaining, by one or more processors, image data associated with a plurality of images of a lesion of a patient positioned in a three-dimensional (3D) space, where the plurality of images are captured from a plurality of imaging devices positioned about the patient;
for each image of the plurality of images, backprojecting, by the one or more processors, points representing the lesion into the 3D space to determine a plurality of distribution confidence values for a subset of voxels within the three-dimensional space;
determining, by the one or more processors, a three-dimensional confidence distribution based on confidence values from the plurality of distribution confidence values corresponding to each voxel of the 3D space; and
determining, by the one or more processors, a position of the lesion within the 3D space based on the 3D confidence distribution.

9. The method of claim 8, further comprising:
determining, by the one or more processors, a position of a radiotherapy system configured to deliver energy relative to at least a portion of the 3D space; and
generating, by the one or more processors, a beam-eye view of the lesion of the patient relative to the radiotherapy system based on the position of the radiotherapy system and the position of the lesion within the 3D space.

10. The method of claim 8, wherein obtaining the image data comprises:
obtaining, by the one or more processors, the image data from the plurality of imaging devices that are positioned in fixed relation to the 3D space, and
in response to registration of a radiotherapy system with the 3D space or the patient, generating, by the one or more processors, a beam-eye view of the lesion of the patient relative to the radiotherapy system.

11. The method of claim 8, wherein obtaining the image data comprises:
obtaining, by the one or more processors, the image data from the plurality of imaging devices that are positioned in fixed relation to a radiotherapy system; and
in response to registration of a radiotherapy system with the 3D space or the patient, generating, by the one or more processors, a beam-eye view of the lesion of the patient relative to the radiotherapy system.

12. The method of claim 8, wherein determining the three-dimensional confidence distribution based on confidence values comprises:
for each voxel of the subset of voxels, multiplying, by the one or more processors, the confidence values corresponding to each voxel established in response to backprojecting the points representing the lesion to establish a confidence value for each voxel, and
normalizing, by the one or more processors, the confidence values for each voxel.

13. The method of claim 8, wherein determining the three-dimensional confidence distribution based on confidence values comprises:
determining, by the one or more processors, a plurality of Gaussian distributions based on the confidence values corresponding to each voxel; and
determining, by the one or more processors, the three-dimensional confidence distribution based on the Gaussian distribution of the confidence values.

14. The method of claim 8, further comprising:
comparing, by the one or more processors the position of the lesion within the 3D space to a treatment plan, the treatment plan; and
generating, by the one or more processors, a set of one or more control signals to adjust a position, a power level, or a leaf sequence of a radiotherapy system with the 3D space, the one or more control signals configured to cause the radiotherapy system to deliver energy to the lesion in accordance with the treatment plan.

15. A non-transitory computer-readable medium storing instructions there on that, when executed by one or more processors, cause the one or more processors to:
obtain image data associated with a plurality of images of a lesion of a patient positioned in a three-dimensional (3D) space, where the plurality of images are captured from a plurality of imaging devices positioned about the patient;
for each image of the plurality of images, backproject points representing the lesion into the 3D space to determine a plurality of distribution confidence values for a subset of voxels within the three-dimensional space;
determine a three-dimensional confidence distribution based on confidence values from the plurality of distribution confidence values corresponding to each voxel of the 3D space; and
determining a position of the lesion within the 3D space based on the 3D confidence distribution.

16. The non-transitory computer-readable medium of claim 15, wherein the instructions further cause the one or more processors to:
determine a position of a radiotherapy system configured to deliver energy relative to at least a portion of the 3D space; and
generate a beam-eye view of the lesion of the patient relative to the radiotherapy system based on the position of the radiotherapy system and the position of the lesion within the 3D space.

17. The non-transitory computer-readable medium of claim 15, wherein the instructions that cause the one or more processors to obtain the image data cause the one or more processors to:
obtain the image data from the plurality of imaging devices that are positioned in fixed relation to the 3D space, and
in response to registration of a radiotherapy system with the 3D space or the patient, generate a beam-eye view of the lesion of the patient relative to the radiotherapy system.

18. The non-transitory computer-readable medium of claim 15, wherein the instructions that cause the one or more processors to obtain the image data cause the one or more processors to:
obtain the image data from the plurality of imaging devices that are positioned in fixed relation to a radiotherapy system; and
in response to registration of a radiotherapy system with the 3D space or the patient, generate a beam-eye view of the patient relative to the radiotherapy system.

19. The non-transitory computer-readable medium of claim 15, wherein the instructions that cause the one or more processors to determine the three-dimensional confidence distribution based on confidence values cause the one or more processors to:
for each voxel of the subset of voxels, multiply the confidence values corresponding to each voxel established in response to backprojecting the points representing the lesion to establish a confidence value for each voxel, and
normalize the confidence values for each voxel.

20. The non-transitory computer-readable medium of claim 15, wherein the instructions that cause the one or more processors configured to determine the three-dimensional confidence distribution based on confidence values cause the one or more processors to:
determine a plurality of Gaussian distributions based on the confidence values corresponding to each voxel; and
determine the three-dimensional confidence distribution based on the Gaussian distribution of the confidence values.
